(19) United States
(12) Patent Application Publication
(10) Pub. No.: US 2007/0004973 A1
(43) Pub. Date: Jan. 4, 2007

(54) Tissue treatment methods

(76) Inventor: Sharon Mi Lyn Tan, Brighton, MA (US)

Correspondence Address: FISH & RICHARDSON PC, P.O. BOX 1022, MINNEAPOLIS, MN, 55440-1022, US

(21) Appl. No.: 11/154,106

(22) Filed: Jun. 15, 2005

(51) Int. Cl. A61B 5/00

(52) U.S. Cl. 600/309

(57) ABSTRACT

Methods of treating tissue are disclosed. The methods can include disposing a plurality of particles including a therapeutic agent within tissue of a subject. The plurality of particles can be exposed to energy to release at least some of the therapeutic agent from the particles. The methods can also include exposing a plurality of particles disposed in a subject to multiple intervals of energy. The energy can release therapeutic agent from at least some of the particles.

### TECHNICAL FIELD

This invention relates to tissue treatment methods.

### BACKGROUND

Therapeutic agents (e.g., drugs) can be used to treat various different medical conditions. For example, certain types of therapeutic agents, generally referred to as anti-cancer agents, can be used to treat cancer.

### SUMMARY

This invention relates to tissue treatment methods.

In one aspect of the invention, a method of treating tissue of a subject includes forming a cavity within the tissue of the subject by disposing a plurality of particles within the tissue of the subject. At least some of the particles include a polymeric material and a therapeutic agent. The plurality of particles are exposed to energy. The energy releases at least some of the therapeutic agent from the particles.

In another aspect of the invention, a method of treating a subject includes exposing a plurality of particles disposed in the subject to multiple intervals of energy. At least some of the particles include a polymeric material and a therapeutic agent. The energy releases at least some of the therapeutic agent from at least some of the particles.

Embodiments can include one or more of the following features.

The method can further include inserting a needle into the tissue, and injecting the particles into the tissue through the needle.

The polymeric material can include poly(glycolic acid), poly(L-lactic acid), polyoxalates, poly(α-esters), polyanhydrides, polyacetates, polycaprolactones, poly(orthoesters), polyamino acids, polyurethanes, polycarbonates, polyiminocarbonates, polyamides, poly (alky cyanoacrylates), stereopolymers of L- and D-lactic acid, copolymers of 1,3bis(p-carboxyphenoxy) propane and sebacic acid, sebacic acid copolymers, copolymers of caprolactone, poly(lactic acid)/poly(glycolic acid)/polethyleneglycol terpolymers, copolymers of polyurethane and poly(lactic acid), copolymers of α-amino acids, copolymers of α-amino acids and caproic acid, copolymers of α-benzyl glutamate and polyethylene glycol, copolymers of poly succinic acid and poly(glycols), polyphosphazene, polyhdroxy-alkanoates, poly(ethylene oxide), poly(ethylene glycol), poly(propylene glycol), poly(ε-caprolactone), poly(α-amino acids), polyurethanes, poly(vinyl alcohol), poly(vinyl pyrrolidone), poly hydroethyl methacrylate, and/or poly hydroxyethyl methacrylate.

The energy can be emitted from a device positioned external to the subject.

The energy can be emitted from a device positioned within the subject.

The energy can be ultrasound energy, UV energy, IR energy, visible light, and/or RF energy.

The energy can be ultrasound energy that has a frequency of from about 20 kHz to about 10 MHz.

The energy can be UV energy, IR energy, and/or visible light. The energy can have have a wavelength of from about 200 nm to about 800 nm.

The therapeutic agent can include an anti-cancer agent.

The method can include exposing the plurality of particles to energy in multiple intervals.

At least some of the particles can include a core and a layer surrounding the core, the layer including the polymeric material and the therapeutic agent.

The core can include polyvinyl alcohol and the layer can include sodium alginate.

The layer can include multiple layers, each of the multiple layers including the polymeric material and the therapeutic agent.

At least some of the multiple layers can include different therapeutic agents.

The energy can be transmitted in multiple intervals to release the agent from the multiple layers.

At least some of the multiple layers can be formed of a bioerodible material.

The core can include a second polymeric material and a second therapeutic agent.

The method can include sequentially exposing the plurality of particles to at least two different forms of energy.

The method can include simultaneously exposing the plurality of particles to at least two different forms of energy.

The method can include exposing the plurality of particles to at least two different intensities of the same energy.

The method can include exposing the plurality of particles to energy at least once a month (e.g., at least once a week, at least once a day, at least twice a day, at least three times a day).

The method can include exposing the plurality of particles to energy for at least about 20 seconds per interval (e.g., at least about one minute per interval, at least about five minutes per interval).

During each interval, at least some the therapeutic agent can be released from at least some of the particles.

The particles can substantially retain the therapeutic agent between the multiple intervals of energy exposure.

Before exposing the plurality of particles to the multiple intervals of energy, a cavity can be formed within a tissue of the subject, and the particle can be disposed within the cavity formed in the tissue.

The method can include inserting a needle into the tissue of the subject to form the cavity, and injecting the particles into the cavity through the needle.

The methods can provide one or more of the following advantages.

In some embodiments, the methods allow an individual (e.g., a physician) to more precisely control the release of therapeutic agent(s) from the particles. For example, the methods can be used to control the timing of the release of the therapeutic agent(s), the quantity of the therapeutic agent(s) released, and/or the chemical constituents contained in the therapeutic agent(s) that are released.

In certain embodiments, the therapeutic agent(s) can be released from the particles in multiple intervals. In some embodiments, the therapeutic agent(s) can be released from the particles multiple times before the therapeutic agent(s) is/are completely expended.

In some embodiments, the therapeutic agent(s) can be released upon exposure to at least two different forms of energy. For example, one form of energy can be used to release one therapeutic agent, and a different form of energy can be used to release a different therapeutic agent. In certain embodiments, the therapeutic agent(s) can be released upon sequential or simultaneous exposure to different forms of energy.

In some embodiments, the therapeutic agent(s) can be released upon exposure to at least two different intensities of the same energy. For example, one energy intensity can be used to release one therapeutic agent, and a different energy intensity can be used to release a different therapeutic agent.

In general, the methods can reduce the frequency with which certain procedures, such as injections, are performed when treating a subject having a medical condition, such as cancer.

Features and advantages are in the description, drawings, and claims.

### DESCRIPTION OF DRAWINGS

FIG. 1 is a cross-sectional view of a cancerous liver of a subject.

FIG. 2 is a cross-sectional view of the liver of FIG. 1 having particles containing a therapeutic agent disposed therein.

FIG. 3 illustrates a method of administering particles into the cancerous tissue of the liver of FIG. 1.

FIG. 4 illustrates a method of transmitting energy to particles disposed within a cancerous tissue region of the liver of FIG. 2 to release the therapeutic agent from the particles.

FIG. 5 illustrates another method of transmitting energy to particles disposed within a cancerous tissue region of the liver of FIG. 2 to release the therapeutic agent from the particles.

Like reference symbols in the drawings indicate like elements.

### DETAILED DESCRIPTION

In general, the methods involve disposing particles, which are formed of a polymeric material and one or more therapeutic agents, within a subject, such as in a portion of the subject's tissue, and exposing the particles to energy to release the therapeutic agent(s).

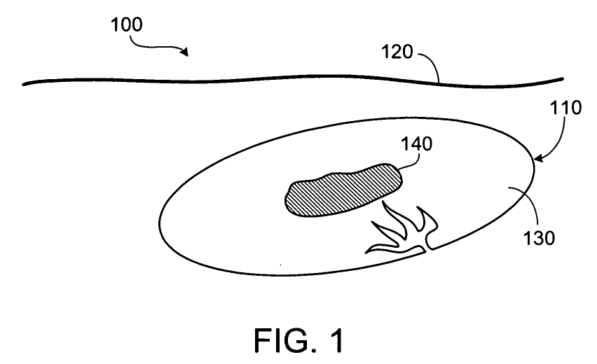

FIG. 1 shows a portion 100 of a subject including a liver 110 and skin 120. The liver 110 includes healthy tissue 130 and unhealthy tissue 140, such as a cancerous tissue (e.g., a cancerous tumor).

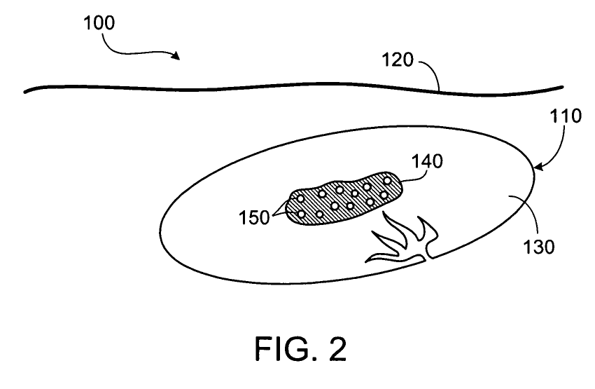

FIG. 2 shows a plurality of particles 150 disposed within unhealthy tissue 140. In general, particles 150 can be distributed homogeneously or non-homogeneously throughout unhealthy tissue 140. Particles 150 are generally spherical and have diameters ranging from about 10 microns to about 3000 microns. Particles 150 are formed of a porous polymeric material, and include one or more anti-cancer agents (e.g., paclitaxel, doxorubicin, cisplatin, and/or carboplatin) that are releasably retained within the pores of the polymeric material of particles 150. As discussed below, the anti-cancer agent(s) can escape from the polymeric material when particles 150 are exposed to ultrasound energy. Without wishing to be bound by theory, it is believed that upon exposing particles 150 to ultrasound, for example, the thermal energy produced by the ultrasound can increase the size of the pores in which the anti-cancer agent(s) is/are stored to release or increase the rate of release of at least some of the anti-cancer agent(s) from particles 150. It is also believed that vibrational energy imparted to particles 150 from the ultrasound can release or increase the release rate of the anti-cancer agent(s) from particles 150.

The rate of release of the anti-cancer agent(s) from particles 150 can depend on the size of the pores within the polymeric structure of particles 150. The rate of release typically increases as the pores increase in size and typically decreases as the pores decrease in size. In certain embodiments, particles 150 are formed of one or more macroporous polymers. Macroporous polymers typically have a pore size in the range of about 500 angstrom to about 1.0 micron (e.g., about 500 angstrom to about 0.75 micron, about 500 angstrom to about 0.5 micron, about 500 angstrom to about 0.25 micron, about 750 angstrom to about 0.75 micron, 0.1 micron to about 0.5 micron).

Alternatively or additionally, particles 150 can be formed of one or more microporous polymers. Microporous polymers typically have a pore size of about 100 angstrom to about 500 angstrom (e.g., about 200 angstrom to about 500 angstrom, about 300 angstrom to about 500 angstrom, about 400 angstrom to about 500 angstrom, about 300 angstrom to about 400 angstrom). The microporous polymer or polymers from which particles 150 are formed, for example, can be loaded with macro molecular anti-cancer agent(s). See, for example, Rhine et al., J. of Pharmaceutical Sci., 69: 265-263 (1980).

Particles 150 can be formed using any of various systems and techniques, such as emulsion polymerization and/or droplet polymerization techniques. Examples of droplet polymerization systems and techniques are described, for example, in co-pending Published Patent Application No. US 2003/0185896 A1, published Oct. 2, 2003, and entitled “Embolization,” and in co-pending Published Patent Application No. US 2004/0096662 A1, published May 20, 2004, and entitled “Embolization,” each of which is incorporated herein by reference.

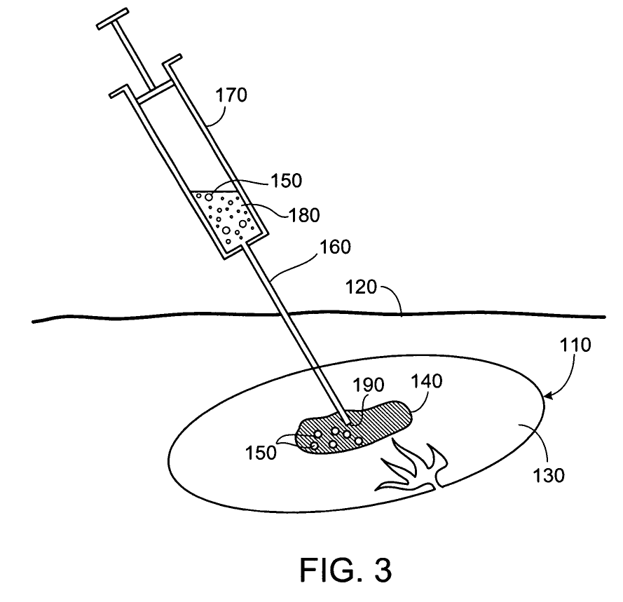

FIG. 3 illustrates a method of disposing particles 150 within unhealthy tissue 140. As shown, a needle 160 can be inserted into unhealthy tissue 140, and particles 150 can be injected through needle 160 into unhealthy tissue 140. Needle 160 is in fluid communication with a syringe 170, which contains multiple particles 150 suspended in a carrier fluid 180. Carrier fluid 180, for example, can be a pharmaceutically acceptable carrier, such as saline, contrast agent, deionized water, water for injection, liquid polymer, gel polymer, gas, therapeutic agent, or a combination of these carriers. An end 190 of needle 160 can be inserted through skin 120, through healthy tissue 130, and into unhealthy tissue 140. After positioning end 190 of needle 160 within unhealthy tissue 140, the composition of particles 150 and carrier fluid 180 can be injected from syringe 170 into unhealthy tissue 140. The composition of particles 150 and carrier fluid 180, for example, can be injected into and contained within a cavity formed by needle 160 or a similar device. Alternatively or additionally, particles 150 and carrier fluid 180, upon being injected into unhealthy tissue 140, can penetrate particular regions of unhealthy tissue 140. For example, the injection of particles 150 and carrier fluid 180 into unhealthy tissue 140 can create a cavity or void within unhealthy tissue 140 that is partially or completely filled by the composition of particles 150 and carrier fluid 180. The above-described penetration of particles 150 into regions of unhealthy tissue 140 can be particularly effective, for example, when using smaller particles (e.g., particles having a diameter of no greater than about 100 microns). However, larger particles, as described below, can also be used.

In certain embodiments, particles 150 are not suspended in a carrier fluid. For example, particles 150 alone can be contained within syringe 170 and injected from syringe 170 into unhealthy tissue 140.

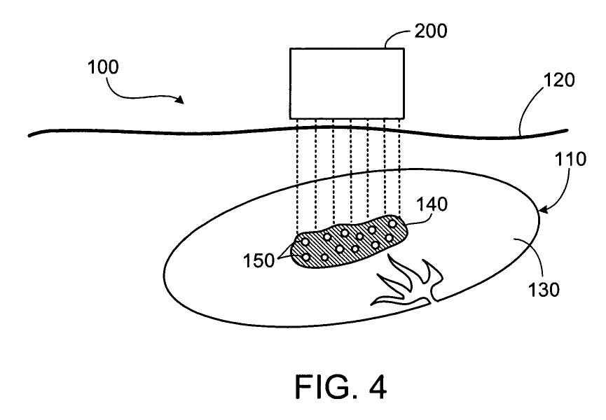

FIG. 4 illustrates a method of transmitting ultrasound energy to particles 150 disposed within unhealthy tissue 140 of a subject. Exposure of particles 150 to ultrasound energy, as noted above, can release or increase the rate of release of the anti-cancer agent(s) from particles 150. As illustrated, an ultrasonic device 200 can be positioned external to the subject (e.g., above skin 120) and activated. The ultrasound, for example, can be transmitted from ultrasonic device 200 through skin 120, through healthy tissue 130, and into unhealthy tissue 140 where it reaches particles 150. It is believed that upon reaching particles 150, the localized heating caused by the ultrasound can increase the size of the pores within the porous polymeric material of particles 150 and cause the release or increase the rate of release of the anti-cancer agent(s) from particles 150. Similarly, it is believed that vibrations caused by the ultrasound can disrupt the polymeric structure of particles 150 and cause the release or increase the rate of release of the anti-cancer agent(s) from particles 150.

The intensity of the ultrasound transmitted from ultrasonic energy device 200 to unhealthy tissue 140 can be a function of the distance between skin 120 and unhealthy tissue 140. For example, as the distance between skin 120 and unhealthy tissue 140 increases, the intensity of the energy used to release the anti-cancer agent(s) can increase. Likewise, as the distance between tissue 140 and skin 120 decreases, the intensity of the energy used to release the anti-cancer agent(s) can decrease. This can be particularly beneficial when unhealthy tissue 140 is situated in relatively close proximity to skin 120. However, such techniques can be used when unhealthy tissue 140 is positioned at any of various depths beneath skin 120. For example, in some embodiments, extra-dermal transmission of ultrasound can be transmitted to particles disposed within tissue about ten centimeters or less (e.g., about five centimeters or less, about four centimeters or less, about three centimeters or less, about three centimeters to about five centimeters) below skin 120.

The release and/or rate of release of the anti-cancer agent(s) from particles 150 can be regulated by varying one or more of the frequency, duration, and intensity of the ultrasound energy. For example, increasing the frequency, duration, and/or intensity of the ultrasound energy can increase the likelihood and/or the rate of release of the anti-cancer agent(s) from particles 150. Similarly, decreasing the frequency, duration, and/or intensity of the ultrasound energy can decrease the likelihood and/or the rate of release of the anti-cancer agent(s) from particles 150. The frequency, duration, and/or intensity can be increased or decreased depending on various factors, such as the depth of unhealthy tissue 140 beneath skin 120 and the targeted dosage of the anti-cancer agent to be released. For example, it may be beneficial to increase the frequency, duration, and/or intensity as the depth of tissue 140 beneath skin 120 and/or the targeted dosage increases. Similarly, it may be beneficial to decrease the frequency, duration, and/or intensity as the depth of tissue 140 beneath skin 120 and/or the targeted dosage decreases.

The frequency of the ultrasound energy transmitted to particles 150 typically can range from about 20 kHz to about ten MHz (e.g., from about 50 kHz to about 200 kHz). Each transmission of the ultrasound energy can last, for example, about ten seconds or longer (e.g, about 20 seconds or longer, about 30 seconds or longer, about 45 seconds or longer, about one minute or longer, about two minutes or longer, about four minutes or longer, about six minutes or longer, about eight minutes or longer, about ten minutes or longer, from about 20 seconds to about ten minutes, from about 20 seconds to about five minutes, from about 20 seconds to about one minute). The intensity of the ultrasound energy can range, for example, from about 0.1 W/cm² to about 30 W/cm² (e.g., from about one W/cm² to about 50 W/cm²). The ultrasound energy can be transmitted to particles 150 in a continuous fashion or in a pulsed fashion.

In some embodiments, the ultrasound energy can be transmitted to particles 150 in multiple intervals in order to release the anti-cancer agent(s) in corresponding intervals. Particles 150, for example, can include a sufficient amount of the anti-cancer agent(s) to allow the release of the anti-cancer agent(s) in response to each of multiple transmissions of ultrasound energy. As a result, the subject can receive multiple treatments with only one injection of particles 150. In some embodiments, the anti-cancer agent(s) can be released from particles 150 at least one time (e.g., at least about two times, at least about four times, at least about six times, at least about eight times, at least about ten times, at least about 20 times, at least about 30 times) before the ultrasound is rendered incapable of releasing anymore anti-cancer agent(s) from particles 150 (e.g., before the anti-cancer agent(s) is/are completely expended from particles 150).

The number and frequency of intervals in which the ultrasound is transmitted to particles 150 can depend on any of various factors, such as the type of medical condition being treated, the severity of the medical condition being treated, and the type of anti-cancer agent(s) being used. The ultrasound energy, for example, can be transmitted to particles 150 at least about one time per month (e.g., at least about three times per month, at least about five times per month, at least about ten times per month, at least about 20 times per month, at least about one time per week, at least about three times per week, at least about five times per week, at least about one time per day, at least about two times per day, at least about three times per day). The ultrasound energy can be transmitted for a predetermined time during the above-noted intervals. For example, the ultrasound energy can be transmitted for at least about ten seconds (e.g., at least about 20 seconds, at least about 30 seconds, at least about 45 seconds, at least about one minute, at least about five minutes, at least about ten minutes) during each of the intervals.

In some embodiments, the ultrasound is transmitted for a common period of time, at a common intensity, and/or at a common frequency for each interval. In certain embodiments, however, the duration, intensity, and/or frequency of energy transmission can increase or decrease as the treatment progresses. For example, it may be beneficial, in some cases, to decrease the duration, intensity, and/or frequency near the end of a treatment cycle (e.g., after a predetermined number of intervals) in order to wean the subject off of the anti-cancer agent(s). Similarly, as the treatment progresses, the subject's need for the anti-cancer agent(s) may decrease and/or negative side effects caused by the anti-cancer agent(s) may increase making it beneficial to decrease the duration, intensity, and/or frequency. For certain treatments, it might be beneficial to ramp up the dosage of anti-cancer agent(s) released. In certain embodiments, the duration, intensity, and/or frequency can be increased after a predetermined number of treatments. In some cases, the duration, intensity, and/or frequency can gradually increase or decrease with each interval.

While certain embodiments have been described above, other embodiments are also possible.

As an alternative to or in addition to ultrasound energy, for example, any of various other forms of energy can be used to release or increase the rate of release of the anti-cancer agent(s) from particles 150. In certain embodiments, the energy includes RF energy, UV energy, IR energy, and/or visible light.

While, in the embodiments discussed above, particles 150 are formed of a porous polymeric material, particles 150 can be formed of any of various other polymeric structures. In some embodiments, particles 150 are formed of nonporous polymers, such as hydrogels. Hydrogels can have internal structure based on molecular chains of entangled, cross-linked, and/or crystalline chain networks in the polymer. The anti-cancer agent(s) can be contained within a space between the molecular chains. The space between macromolecular chains of hydrogels is referred to as the mesh size. Examples of hydrogels include polyhydroxyethylmethacrylate, polyvinyl alcohol, polyanhydrides, polyglycolides, and polylactides.

In certain embodiments, particles 150 are formed of cross-linked polymer chains, which can produce a screening effect to releasably retain the anti-cancer agent(s) within the particles. In some embodiments, when particles formed of cross-linked polymer chains are exposed to certain types of energy, such as ultrasound and/or RF energy, the energy can cause the cross-linked structure to degrade resulting in the release of the anti-cancer agent(s) from the particles. For example, the cross-linked polymer can include bonds that break upon exposure to localized elevated temperatures produced by the energy. Examples of such bonds include ester or acids with amine introduced into the polymer by side chain reactions. In some embodiments, the vibrations produced by certain types of energy, such as ultrasound, can break bonds within the polymeric structure. The vibrations, for example, can cause one or more of the polymer chains to become cleaved. Consequently, the anti-cancer agent(s) can be released from particles 150. Examples of polymeric materials suitable for use in this embodiment include, but are not limited to, poly(L-lysine-co-polyethyleneglycol), poly(methacrylic acid-co-methacryloxyethylglucoside) and poly(methacrylic acid-co-ethyleneglycol), polylactic acid (PLA), polyglycolic acid (PGA), polyamides, poly(ε-caprolactone), poly(orthoesters), and polyanhydrides. Further non-limiting examples of suitable polymers in forming the coating include polyanhydrides, ethylene-vinyl acetate, poly(lactic acid), poly(glutamic acid), poly(ε-caprolactone), lactic/glycolic acid copolymers, polyorthoesters, polyamides and the like. Any of various cross-linking agents can be used.

In certain embodiments, particles 150 are formed of entangled polymeric chains that can similarly be exposed to particular forms of energy to create a localized temperature increase and/or vibrations that can cause the release or increase the rate of release of the anti-cancer agent(s) from particles 150. The heat and/or vibrations caused by ultrasound and/or RF energy, for example, can increase the mesh size of the entangled polymeric structure to release or increase the rate of release of the anti-cancer agent(s) from particles 150.

In some embodiments, particles 150 can be formed of one or more polymeric materials including a pendant group that can be solubilized. Solubilization of water-insoluble polymers, for example, can occur as a result of hydrolysis, ionization, or protonation of a side group. When particles formed of such materials are exposed to certain types of energy, such as ultrasound, the energy can cause the release or increase the rate of release of the anti-cancer agent(s) from the particle. Polymers of this type include, for example, poly(L-lysine-co-polyethyleneglycol), poly(methacrylic acid-co-methacryloxyethylglucoside), and poly(methacrylic acid-co-ethyleneglycol).

In certain embodiments, particles 150 are formed of one or more high molecular weight water-insoluble polymers having labile bonds in the polymer backbone. Upon exposure to certain types of energy, these labile bonds can become cleaved, and the cleaved portion of the polymer can be converted into small, water-soluble molecules. This can cause the release or increase the rate of release of the anti-cancer agent(s) from particles 150. Alternatively or additionally, a percolation technique can break the backbone bonds causing the volume of the polymer to increase and allowing the anti-cancer agent(s) captured therein to flow out of particles 150. Examples of polymers that can be used in this embodiment include polylactic acid (PLA), polyglycolic acid (PGA) and lactic/glycolic acid co-polymer, polyamides, poly(ε-caprolactone), poly(orthoesters), and polyanhydrides. Further examples of polymers that can be used in this embodiment include polyanhydrides, ethylene-vinyl acetate, poly(lactic acid), poly(glutamic acid), poly(ε-caprolactone), lactic/glycolic acid copolymers, polyorthoesters, and polyamides.

In some embodiments, particles 150 are formed of a polymeric structure including a reservoir system in which a polymeric membrane surrounds a core of anti-cancer agent(s). In this embodiment, a porous or non-porous polymer encapsulates anti-cancer agent(s) within micro- or nano-particles, which form micro-containers or micelles for the anti-cancer agent(s). Non-limiting examples of polymers that can be used in this embodiment include poly(ethylene glycol) (PEG), poly(acrylic acid) (PAA) and poly(vinyl alcohol) (PVA) or co-polymers or block polymers thereof. See, for example, Tian and Uhrich, Polymer Preprints, 43(2): 719-720 (2002). The polymer can be amphiphilic, containing controlled hydrophobic and hydrophilic balance (HLB), which can facilitate organization of the polymer into circular micelles. When particles including such a reservoir system are exposed to certain types of energy, such as ultrasound and/or RF energy, the energy can alter the polymeric structure to release at least some of the anti-cancer agent(s) from particles 150. Examples of suitable polymers with which reservoir systems include hydrogels such as swollen poly(2-hydroxyethyl methacrylate) (PEMA), silicone networks, and ethylene vinyl acetate copolymers. Further examples include polyvinyl alcohol, polyvinyl pyrrolidone, and polyethylene oxide. Other polymers can also be used. See, for example, Pedley et al., Br. Polymer J., 12: 99 (1980).

In certain embodiments, the polymeric material of particles 150 includes micelles that surround the anti-cancer agent(s). The micelles, for example, can include air bubbles. The micelles can have a diameter of about 0.01 micron to about 100 microns and a gas volume of about 5% to about 30% of the volume of the micelles. Application of ultrasound to particles 150 can cause the air bubbles in the micelles to pulsate. As a result, the air bubbles can become asymmetric at the air/liquid interface. The surface of such a pulsating asymmetric oscillation bubble can cause a steady eddying motion to be generated in the immediate adjoining liquid, often called microstreaming. This pulsating results in localized shearing action that can be strong enough to cause fragmentation of internal structures of the polymer. For example, main chain rupture may be induced by shock waves during cavitiation of the liquid medium.

In certain embodiments, particles 150 are formed of one or more polymers including a photosensitizer linked to the backbone or side chain of the backbone of the polymer. When exposed to a particular energy (e.g., light energy having a wavelength between about 200 nm and 800 nm), the polymer can release the anti-cancer agent(s). In some embodiments, the anti-cancer agent(s) may be linked via a side chain to the polymer backbone, and the photosensitzer may be linked to the same or different polymer backbone in the vicinity of the anti-cancer agent(s). It is also possible to attach a photosensitizer directly to the anti-cancer agent(s), or to interpose a photosensitizer between a linker and the anti-cancer agent(s). Examples of polymers that can be used in these embodiments include co-polymers of N-(-2 hydroxypropyl)methacrylamide and an enzymatically degradable oligopeptide poly (L-lysine-copolyethylene glycol).

In certain embodiments, particles 150 include a photoreactive compound or photosensitizer linked to a polymer backbone using an appropriate linker, which can release the anti-cancer agent(s) upon being exposed to certain types of energy, such as UV energy, IR energy, and/or visible light. For example, photosensitizers can be bound to anti-cancer agent(s) having aliphatic amino groups to form photoreactive/anti-cancer agent complexes. Polymer backbones or co-polymer precursors, for example, may be derivatized to contain co-polymer side chains or “linkers” having active ester functionalities. The aliphatic amino groups of the complexes may be bound to the active ester functionalities of the linker by aminolysis reactions. These stable moieties may be formed into co-polymers to be used in the formation of particles 150. Application of an appropriate form of energy, for example, can result in release of the anti-cancer agent(s) from the polymer by breaking a bond to the linker. See, for example, N. L. Krinick et al., J. Biomater. Sci. Polymer Edn., 5(4): 303-324 (1994). The photochemically reactive group can be furfuryl alcohol or meso-chlorin e6 monoethylene diamine disodium salt.

Photoreactive agents may be used in conjunction with one or more anti-cancer agents linked to the polymeric material of particles 150. The release of the anti-cancer agents can be controlled, for example, by exposure of particles 150 to UV energy, IR energy, and/or visible light. Examples of polymers that can be used in this embodiment include copolymers of N(-2-hydroxypropyl)methacrylamide and a linker, such as poly(L-lysine-co-polyethylene glycol). Further, non-limiting examples of suitable polymers for this embodiment include poly(propylene glycol) (PPG), poly(vinyl alcohol) (PVA) and poly(acrylic acid) (PAA).

Photosensitizers useful for attachment to one or more anti-cancer agents or linkers can include dabcyl succinimidyl ester, dabcyl sulfonyl chloride, malachite green isothiocyanate, QSY7 succinimidyl ester, SY9 succinimidyl ester, SY21 carboxylic acid succinimidyl ester, and/or SY35 acetic acid succinimidyl ester, which are commercially available from Invitrogen Life Sciences, Carlsbad, Calif. These photoreactive agents can absorb light in the range of from about 450 nm to about 650 nm.

In some embodiments, particles 150 include a polymeric material and one or more anti-cancer agents joined by a linking moiety. The linking moiety can be attached at a first end to the polymeric material and at a second end via a photochemically reactive group to the anti-cancer agent(s). See, for example, U.S. Pat. Nos. 5,263,992 and 6,179,817, which are incorporated herein by reference. Exposure to UV energy, IR energy, and/or visible light, for example, can cause the photochemically reactive group to release the anti-cancer agent(s).

In certain embodiments, anti-cancer agents having, or derivatized to contain, reactive aliphatic amino groups can be bound to polymers having, or derivatized to contain, ester or acid functional groups. The ester or acid moieties, for example, may be present on a polymer or co-polymer side chain. Amidization reaction can bind the aliphatic amino groups of the anti-cancer agent to the ester groups on the polymer.

In certain embodiments, particles 150 include a linker having a photoreactive group arranged between a polymeric material and an anti-cancer agent. The photoreactive group and the anti-cancer agent may be embedded in the polymeric material or coated on a surface thereof. The photoreactive group, for example, can release the anti-cancer agent upon exposure to light in the wavelength range of from about 200 nm to about 800 nm.

In some embodiments, the polymeric material used to form particles 150 includes both bonds and pores that react upon exposure to ultrasound energy so as to release the anti-cancer agent(s).

In certain embodiments, particles 150 can be coated with one or more of the polymers or polymer systems discussed above, which can contain one or more anti-cancer agents. Particles 150, for example, can include a polyvinyl alcohol matrix polymer surrounded by a sodium alginate coating that contains the anti-cancer agent(s). The polymeric material of the coating can be adapted to controllably release the anti-cancer agents upon exposure to one or more forms of energy. Particles having coatings are disclosed, for example, in commonly owned and co-pending Patent Application Publication No. US 2004-0076582 A1, published on Apr. 22, 2004, and in commonly owned and co-pending patent application Ser. No. 10/858,253, which are incorporated herein by reference.

While some examples of polymeric materials from which particles 150 can be formed were described above, one or more of the polymeric materials listed below can alternatively or additionally be used. For example, particles 150 can be formed of poly(glycolic acid) (PGA), poly(L-lactic acid) (PLLA) (PLA), polyoxalates, poly(α-esters), polyanhydrides, polyacetates, polycaprolactones, poly(orthoesters), polyamino acids, polyurethanes, polycarbonates, polyiminocarbonates, polyamides, poly (alky cyanoacrylates), and mixtures and copolymers thereof. Additional examples of polymeric materials include, stereopolymers of L- and D-lactic acid, copolymers of 1,3bis(p-carboxyphenoxy)propane and sebacic acid, sebacic acid copolymers, copolymers of caprolactone, poly(lactic acid)/poly(glycolic acid)/polethyleneglycol terpolymers, copolymers of polyurethane and poly(lactic acid), copolymers of α-amino acids, copolymers of α-amino acids and caproic acid, copolymers of α-benzyl glutamate and polyethylene glycol, copolymers of poly succinic acid and poly(glycols), polyphosphazene, polyhdroxy-alkanoates and mixtures thereof.

In some embodiments, particles 150 are formed of poly(ethylene oxide) (PEO), poly(ethylene glycol) (PEG), poly(propylene glycol) (PPG), poly (L-lactic acid) (PLLA), poly(ε-caprolactone), poly(α-amino acids), polyurethanes, poly(vinyl alcohol) (PVA) poly(vinyl pyrrolidone), poly hydroethyl methacrylate, polyhydroxyethyl methacrylate, and/or copolymers thereof.

Examples of additional polymers from which particles 150 may be formed include poly (lactic acid-co-glycolic acid) (PLGA), poly (lactic acid-co-ε-caprolactone) (PLACL), PLA-PEG diblock copolymer, PLA-PEG-PLA triblock copolymer, poly (orthoesters), poly (sebactic anhydride), poly(acrylic acid) (PAA) and derivatives, poly(ethylene-co-vinylacetate) (PEVAc), poly (lysine), poly (lactic acid-co-lysine), polyurethanes and block copolymers (e.g., commercially available polyurethanes, k such as BIOMER, ACUTHANE (available from Dow Chemical Co., PELLETHANE (available from Dow Chemical Co., Wilmington, Del.), and RIMPLAST), and poly(dimethylsiloxanes)

Further examples of commercially available polymers that can be used to form particles 150 include PLURONIC (available from BASF Corp., Ludwigshafen, Germany); MEDISORB, ELVAX40P (ethylene vinyl acetate) and BIODEL (available from Dupont Corp., Wilmington, Del.); and Polymer No. 6529C (Poly(lactic acid)) and Polymer No. 6525 (poly(glycolic acid)) available from Polysciences Inc., Warrington, Pa.

The size of particles 150 need not be limited to the sizes discussed above. In certain embodiments, particles 150 have a diameter of no greater than about 10,000 microns (e.g., no greater than about 7,500 microns, no greater than about 5,000 microns, no greater than about 2,500 microns, no greater than about 2,000 microns, no greater than about 1,5000 microns, no greater than about 1,000 microns, no greater than about 500 microns, no greater than about 400 microns, no greater than about 300 microns, no greater than about 200 microns, no greater than about 100 microns). In some embodiments, particles 150 have a diameter of about 100 microns to about 10,000 microns (e.g., about 100 microns to about 1000 microns, about 100 microns to 500 microns, about 2,500 microns to about 5,000 microns, about 5,000 microns to about 10,000 microns, about 7,500 microns to about 10,000 microns).

While the particles described above are typically formed to be spherical, they can also be non-spherical. Non-spherical particles, for example, can be produced using techniques similar to those described above. Non-spherical particles can be manufactured and formed, for example, by controlling drop formation conditions. In some embodiments, non-spherical particles can be formed by post-processing the particles (e.g., by cutting or dicing into other shapes). Particle shaping is described, for example, in co-pending Published Patent Application No. US 2003/0203985 A1, published on Oct. 30, 2003, and entitled “Forming a Chemically Cross-Linked Particle of a Desired Shape and Diameter,” which is incorporated herein by reference.

While the embodiments described above involve particles including anti-cancer agent(s), any therapeutic agent can alternatively or additionally be used. Therapeutic agents include agents that are negatively charged, positively charged, amphoteric, or neutral. Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; gene therapies; nucleic acids with and without carrier vectors; oligonucleotides; gene/vector systems; DNA chimeras; compacting agents (e.g., DNA compacting agents); viruses; polymers; hyaluronic acid; proteins (e.g., enzymes such as ribozymes); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Non-limiting examples of therapeutic agents include anti-thrombogenic agents; antioxidants; angiogenic and anti-angiogenic agents and factors; anti-proliferative agents (e.g., agents capable of blocking smooth muscle cell proliferation); calcium entry blockers; and survival genes which protect against cell death.

Exemplary non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, sulfasalazine and mesalamine; antineoplastic/antiproliferative/anti-mitotic agents such as paclitaxel, 5-fluorouracil, cisplatin, doxorubicin; vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; vascular cell growth promoters such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Exemplary genetic therapeutic agents include: anti-sense DNA and RNA; DNA coding for: anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Vectors of interest for delivery of genetic therapeutic agents include: Plasmids, Viral vectors such as adenovirus (AV), adenoassociated virus (AAV) and lentivirus, Non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents appropriate for the practice of the present invention are disclosed in U.S. Pat. No. 5,733,925, which is incorporated herein by reference. Therapeutic agents disclosed in this patent include the following: “Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

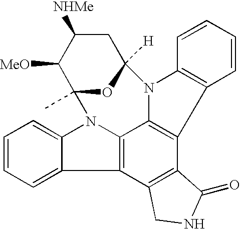

as well as diindoloalkaloids having one of the following general structures:

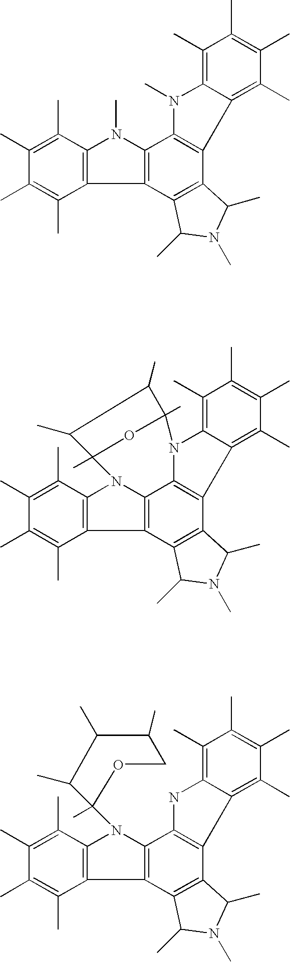

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like.

Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell) such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Examples of such agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents are appropriate for the practice of the present invention and include one or more of the following:
Calcium-channel blockers including:
   Benzothiazapines such as diltiazem and clentiazem
   Dihydropyridines such as nifedipine, amlodipine and nicardapine
   Phenylalkylamines such as verapamil
Serotonin pathway modulators including:
   5-HT antagonists such as ketanserin and naftidrofuryl
   5-HT uptake inhibitors such as fluoxetine
Cyclic nucleotide pathway agents including:
   Phosphodiesterase inhibitors such as cilostazole and dipyridamole
   Adenylate/Guanylate cyclase stimulants such as forskolin
   Adenosine analogs
Catecholamine modulators including:
   α-antagonists such as prazosin and bunazosine
   β-antagonists such as propranolol
   α/β-antagonists such as labetalol and carvedilol
Endothelin receptor antagonists
Nitric oxide donors/releasing molecules including:
   Organic nitrates/nitrites such as nitroglycerin, isosorbide dinitrate and amyl nitrite
   Inorganic nitroso compounds such as sodium nitroprusside
   Sydnonimines such as molsidomine and linsidomine
   Nonoates such as diazenium diolates and NO adducts of alkanediamines
   S-nitroso compounds including low molecular weight compounds (e.g., S-nitroso derivatives of captopril, glutathione and N-acetyl penicillamine), high molecular weight compounds (e.g., S-nitroso derivatives of proteins, peptides, oligosaccharides, polysaccharides, synthetic polymers/oligomers and natural polymers/oligomers)
   C-nitroso-, O-nitroso- and N-nitroso-compounds
   L-arginine
ACE inhibitors such as cilazapril, fosinopril and enalapril
ATII-receptor antagonists such as saralasin and losartin
Platelet adhesion inhibitors such as albumin and polyethylene oxide
Platelet aggregation inhibitors including:
   Aspirin and thienopyridine (ticlopidine, clopidogrel)
   GP Ib/IIIa inhibitors such as abciximab, epitifibatide and tirofiban
Coagulation pathway modulators including:
   Heparinoids such as heparin, low molecular weight heparin, dextran sulfate and β-cyclodextrin tetradecasulfate
   Thrombin inhibitors such as hirudin, hirulog, PPACK(D-phe-L-propyl-L-arg-chloromethylketone) and argatroban
   FXa inhibitors such as antistatin and TAP (tick anticoagulant peptide)
   Vitamin K inhibitors such as warfarin
   Activated protein C
Cyclooxygenase pathway inhibitors such as aspirin, ibuprofen, flurbiprofen, indomethacin and sulfinpyrazone
Natural and synthetic corticosteroids such as dexamethasone, prednisolone, methprednisolone and hydrocortisone
Lipoxygenase pathway inhibitors such as nordihydroguairetic acid and caffeic acid
Leukotriene receptor antagonists
Antagonists of E- and P-selectins
Inhibitors of VCAM-1 and ICAM-1 interactions
Prostaglandins and analogs thereof including:
   Prostaglandins such as PGE1 and PGI2
   Prostacyclin analogs such as ciprostene, epoprostenol, carbacyclin, iloprost and beraprost
Macrophage activation preventers including bisphosphonates
HMG-CoA reductase inhibitors such as lovastatin, pravastatin, fluvastatin, simvastatin and cerivastatin
Fish oils and omega-3-fatty acids
Free-radical scavengers/antioxidants such as probucol, vitamins C and E, ebselen, trans-retinoic acid and SOD mimics
Agents affecting various growth factors including:
FGF pathway agents such as bFGF antibodies and chimeric fusion proteins
PDGF receptor antagonists such as trapidil
IGF pathway agents including somatostatin analogs such as angiopeptin and ocreotide
TGF-β pathway agents such as polyanionic agents (heparin, fucoidin), decorin, and TGF-β antibodies
EGF pathway agents such as EGF antibodies, receptor antagonists and chimeric fusion proteins
TNF-α pathway agents such as thalidomide and analogs thereof.
Thromboxane A2 (TXA2) pathway modulators such as sulotroban, vapiprost, dazoxiben and ridogrel
Protein tyrosine kinase inhibitors such as tyrphostin, genistein and quinoxaline derivatives
MMP pathway inhibitors such as marimastat, ilomastat and metastat
Cell motility inhibitors such as cytochalasin B
Antiproliferative/antineoplastic agents including:
   Antimetabolites such as purine analogs(6-mercaptopurine), pyrimidine analogs (e.g., cytarabine and 5-fluorouracil) and methotrexate
   Nitrogen mustards, alkyl sulfonates, ethylenimines, antibiotics (e.g., daunorubicin, doxorubicin), nitrosoureas and cisplatin
   Agents affecting microtubule dynamics (e.g., vinblastine, vincristine, colchicine, paclitaxel and epothilone)
   Caspase activators
   Proteasome inhibitors
   Angiogenesis inhibitors (e.g., endostatin, angiostatin and squalamine)
   Rapamycin, cerivastatin, flavopiridol and suramin
Matrix deposition/organization pathway inhibitors such as halofuginone or other quinazolinone derivatives and tranilast
Endothelialization facilitators such as VEGF and RGD peptide
Blood rheology modulators such as pentoxifylline.

Therapeutic agents are described, for example, in co-pending Published Patent Application No. US 2004/0076582 A1, published on Apr. 22, 2004, and entitled “Agent Delivery Particle”, which is incorporated herein by reference, and in Pinchuk et al., U.S. Pat. No. 6,545,097, which is incorporated herein by reference.

In some embodiments, particles 150 include a combination of two or more of the above-noted therapeutic agents.

In certain embodiments, particles 150 include a core region and multiple layers surrounding the core region. One or more of the layers (e.g., coatings) can be, for example a degradable and/or bioabsorbable polymer. The coating can be applied by dipping or spraying the particles. The erodible polymer can be a polysaccharide (such as an alginate) or a polysaccharide derivative. In certain embodiments, the coating can be an inorganic, ionic salt. Other erodible coatings include water soluble polymers (such as a polyvinyl alcohol, e.g., that has not been cross-linked), biodegradable poly DL-lactide-poly ethylene glycol (PELA), hydrogels (e.g., polyacrylic acid, haluronic acid, gelatin, carboxymethyl cellulose), polyethylene glycols (PEG), chitosan, polyesters (e.g., polycaprolactones), and poly(lactic-co-glycolic) acids (e.g., poly(d-lactic-co-glycolic) acids).

In some embodiments, energy can be transmitted to particle 150 in multiple intervals to release the therapeutic agent(s) from the multiple layers of coating, respectively. After causing the release of the therapeutic agent(s) from the outermost layer, for example, the outermost layer can erode. As a result, the next transmission of energy can cause the release of the therapeutic agent(s) from the next outermost layer without impedance of the outermost layer that has eroded. In certain embodiments, some of the multiple layers can include different therapeutic agents such that sequential exposures to energy can be used to release different types of therapeutic agents. For example, some of the layers can include one or more therapeutic agents used to treat one medical condition, and some of the other layers can include one or more therapeutic agents used to treat another medical condition.

In some embodiments, particles 150 can include a core that includes one or more therapeutic agents and a coating that includes one or more therapeutic agents. The therapeutic agent(s) in the coating can be the same as or different than the therapeutic agent(s) in the core. Energy can be transmitted to particles 150 to release the therapeutic agents of the core and coating simultaneously or sequentially. Examples of particles having one or more therapeutic agents in a core and in one or more layers surrounding the core (e.g., one ore more coatings) can be found, for example, in commonly owned and co-pending Patent Application Publication No. US 2004-0076582 A1, published on Apr. 22, 2004, and in commonly owned and co-pending patent application Ser. No. 10/858,253, which are incorporated herein by reference.

As an alternative to or in addition to the techniques described above for delivering particles 150 to unhealthy tissue 140, other techniques can be used. In certain embodiments, particles 150 are delivered to unhealthy tissue 140 via a catheter. For example, the catheter can be connected to a syringe barrel with a polymer. The catheter can be inserted into a femoral artery of the subject. Particles or particle compositions can then be injected into the subject's bloodstream in order to deliver the particles to a desired location within the subject.

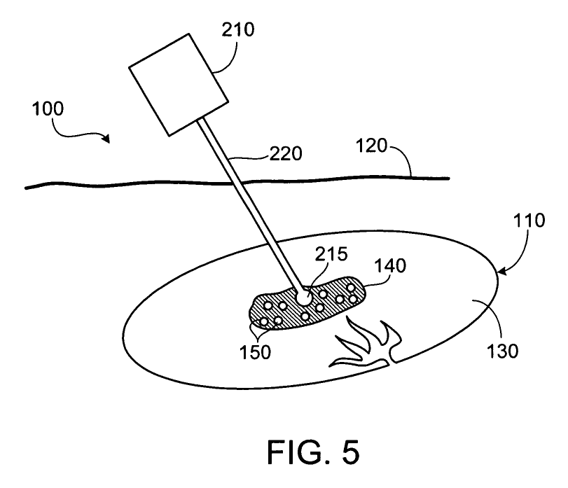

While the methods described above included the use of an extra-dermal energy device, other types of energy devices can be used. For example, energy can be transmitted to particles 150 by a local energy device. As shown in FIG. 5, a local energy device 210 can be inserted into unhealthy tissue 140 and activated. In certain embodiments, energy device 210 includes an energy emitting end 215. Energy device 210 can be inserted through skin 120 and into unhealthy tissue 140 via a cannula 220, for example. Once an end of cannula 220 is positioned within unhealthy tissue 140, energy-emitting end 215 can be deployed from cannula 220 and into unhealthy tissue 140. Energy device 210 can then be activated to emit energy from end 215. The energy can contact particles 150 to release the therapeutic agent(s) contained therein. Use of local energy device 210, for example, can allow energy to be transmitted to particles 150 with substantially undiminished intensity. It may also be advantageous, for example, to use local energy device 210 when tissue 140 is located at a relatively great depth beneath skin 120. Further, it may be beneficial to use local energy device 210 for transmitting particular forms of energy, such as UV energy, IR energy, and visible light, that may be less able to penetrate multiple layers of skin and/or tissue.

In certain embodiments, some of particles 150 include a therapeutic agent that can be released when exposed to a first type of energy, such as, for example, ultrasound, and some of particles 150 include a therapeutic agent that can be released when exposed to a second type of energy, such as, for example, visible light. In some embodiments, some of particles 150 include a therapeutic agent that can be released when exposed to ultrasound, and some of particles 150 include a therapeutic agent that can be released when exposed to UV energy. In some embodiments, some of particles 150 include a therapeutic agent that can be released when exposed to ultrasound, and some of particles 150 include a therapeutic agent that can be released when exposed to IR energy. In some embodiments, some of particles 150 include a therapeutic agent that can be released when exposed to ultrasound, and some of particles 150 include a therapeutic agent that can be released when exposed to RF energy. In some embodiments, some of particles 150 include a therapeutic agent that can be released when exposed to RF energy, and some of particles 150 include a therapeutic agent that can be released when exposed to visible light. In some embodiments, some of particles 150 include a therapeutic agent that can be released when exposed to RF energy, and some of particles 150 include a therapeutic agent that can be released when exposed to UV energy. In some embodiments, some of particles 150 include a therapeutic agent that can be released when exposed to RF energy, and some of particles 150 include a therapeutic agent that can be released when exposed to IR energy. In some embodiments, some of particles 150 include a therapeutic agent that can be released when exposed to visible light, and some of particles 150 include a therapeutic agent that can be released when exposed to UV energy. In some embodiments, some of particles 150 include a therapeutic agent that can be released when exposed to visible light, and some of particles 150 include a therapeutic agent that can be released when exposed to IR energy. In some embodiments, some of particles 150 include a therapeutic agent that can be released when exposed to IR energy, and some of particles 150 include a therapeutic agent that can be released when exposed to UV energy.

Similarly, in some embodiments, some of particles 150 include a therapeutic agent that can be released when exposed to a first intensity of energy, and some of particles 150 include a therapeutic agent that can be released when exposed to a second intensity of the same form of energy.

In some embodiments, particles 150 include different types of therapeutic agents such that particles 150 can be exposed to a first form and/or intensity of energy to treat one type of medical condition, and can be exposed to a second form and/or intensity of energy to treat another type of medical condition. For example, some of particles 150 can include anti-cancer agent(s) and some of particles 150 can include pain-relieving agent(s). Such particles, for example, can be exposed to a first form and/or intensity of energy to release the anti-cancer agent(s), and to a second form and/or intensity of energy to release the pain-relieving agent(s).

While many of the methods discussed above relate to cancer treatments, any of various other medical conditions can be treated using similar methods. The methods can be used to treat medical conditions occurring in any of various organs and tissues including, for example, heart, lung, brain, liver, skeletal muscle, smooth muscle, kidney, bladder, intestines, stomach, pancreas, ovary, prostate, eye, tumors, cartilage, and bone. Furthermore, the tissue to be treated need not be unhealthy tissue. The methods described above can be used to treat any of various types of healthy tissue.

In some embodiments, the particles can include other materials. For example, the particles can include (e.g., encapsulate) diagnostic agent(s) such as a radiopaque material, an MRI-visible material, a ferromagnetic material, and/or an ultrasound contrast agent. In some embodiments, surface preferential material 14 can include one or more of these diagnostic agents. Diagnostic agents are described, for example, in U.S. patent application Ser. No. 10/651,475, filed on Aug. 29, 2003, and entitled “Embolization”, which is incorporated herein by reference.

In certain embodiments, one or more of the particles can include a super-absorbable polymer and/or a shape-memory material (e.g., a polymer). Examples of super-absorbable polymers include Merocel® polymer. Examples of shape-memory materials include nitinol. Shape memory materials and particles that include shape memory materials are described in, for example, U.S. patent application Ser. No. 10/700,970, filed Nov. 4, 2003, and entitled “Embolic Compositions”, and U.S. patent application Ser. No. 10/791,103, filed Mar. 2, 2004, and entitled “Embolic Compositions”, both of which are incorporated herein by reference.

Other embodiments are in the claims.

What is claimed is:

1. A method of treating tissue of a subject, the method comprising:
forming a cavity within the tissue of the subject by disposing a plurality of particles within the tissue of the subject, at least some of the particles comprising a polymeric material and a therapeutic agent; and
exposing the plurality of particles to energy, the energy releasing at least some of the therapeutic agent from the particles.

2. The method of claim 1, further comprising inserting a needle into the tissue, and injecting the particles into the tissue through the needle.

3. The method of claim 1, wherein the polymeric material comprises one or more materials selected from the group consisting of poly(glycolic acid), poly(L-lactic acid), polyoxalates, poly(α-esters), polyanhydrides, polyacetates, polycaprolactones, poly(orthoesters), polyamino acids, polyurethanes, polycarbonates, polyiminocarbonates, polyamides, poly (alky cyanoacrylates), stereopolymers of L- and D-lactic acid, copolymers of 1,3bis(p-carboxyphenoxy) propane and sebacic acid, sebacic acid copolymers, copolymers of caprolactone, poly(lactic acid)/poly(glycolic acid)/polethyleneglycol terpolymers, copolymers of polyurethane and poly(lactic acid), copolymers of α-amino acids, copolymers of α-amino acids and caproic acid, copolymers of α-benzyl glutamate and polyethylene glycol, copolymers of poly succinic acid and poly(glycols), polyphosphazene, polyhdroxy-alkanoates, poly(ethylene oxide), poly(ethylene glycol), poly(propylene glycol), poly(ε-caprolactone), poly(α-amino acids), polyurethanes, poly(vinyl alcohol), poly(vinyl pyrrolidone), poly hydroethyl methacrylate, and poly hydroxyethyl methacrylate.

4. The method of claim 1, wherein the energy is emitted from a device positioned external to the subject.

5. The method of claim 1, wherein the energy is emitted from a device positioned within the subject.

6. The method of claim 1, wherein the energy is selected from the group consisting of ultrasound energy, UV energy, IR energy, visible light, and RF energy.

7. The method of claim 6, wherein the energy comprises ultrasound energy.

8. The method of claim 7, wherein the ultrasound energy has a frequency of from about 20 kHz to about 10 MHz.

9. The method of claim 6, wherein the energy is selected from the group consisting of UV energy, IR energy, and visible light.

10. The method of claim 9, wherein the energy has a wavelength of from about 200 nm to about 800 nm.

11. The method of claim 1, wherein the therapeutic agent comprises an anti-cancer agent.

12. The method of claim 1, wherein the method includes exposing the plurality of particles to energy in multiple intervals.

13. The method of claim 1, wherein at least some of the particles comprise a core and a layer surrounding the core, the layer comprising the polymeric material and the therapeutic agent.

14. The method of claim 13, wherein the core comprises polyvinyl alcohol and the layer comprises sodium alginate.

15. The method of claim 13, wherein the layer comprises multiple layers, each of the multiple layers comprising the polymeric material and the therapeutic agent.

16. The method of claim 15, wherein at least some of the multiple layers comprise different therapeutic agents.

17. The method of claim 15, wherein the energy is transmitted in multiple intervals to release the agent from the multiple layers.

18. The method of claim 17, wherein at least some of the multiple layers are formed of a bioerodible material.

19. The method of claim 13, wherein the core comprises a second polymeric material and a second therapeutic agent.

20. The method of claim 1, wherein the method includes sequentially exposing the plurality of particles to at least two different forms of energy.

21. The method of claim 1, wherein the method includes simultaneously exposing the plurality of particles to at least two different forms of energy.

22. The method of claim 1, wherein the method includes exposing the plurality of particles to at least two different intensities of the same energy.

23. A method of treating a subject, the method comprising:
exposing a plurality of particles disposed in the subject to multiple intervals of energy, at least some of the particles comprising a polymeric material and a therapeutic agent, the energy releasing at least some of the therapeutic agent from at least some of the particles.

24. The method of claim 23, wherein the method includes exposing the plurality of particles to energy at least once a month.

25. The method of claim 23, wherein the method includes exposing the plurality of particles to energy for at least about 20 seconds per interval.

26. The method of claim 23, wherein, during each interval, at least some the therapeutic agent is released from at least some of the particles.

27. The method of claim 23, wherein the particles substantially retain the therapeutic agent between the multiple intervals of energy exposure.

28. The method of claim 23, further comprising, before exposing the plurality of particles to the multiple intervals of energy, forming a cavity within a tissue of the subject, and disposing the particle within the cavity formed in the tissue.

29. The method of claim 28, wherein the method includes inserting a needle into the tissue of the subject to form the cavity, and injecting the particles into the cavity through the needle.

30. The method of claim 23, wherein the polymeric material comprises one or more materials selected from the group consisting of poly(glycolic acid), poly(L-lactic acid), polyoxalates, poly(α-esters), polyanhydrides, polyacetates, polycaprolactones, poly(orthoesters), polyamino acids, polyurethanes, polycarbonates, polyiminocarbonates, polyamides, poly (alky cyanoacrylates), stereopolymers of L- and D-lactic acid, copolymers of 1,3bis(p-carboxyphenoxy) propane and sebacic acid, sebacic acid copolymers, copolymers of caprolactone, poly(lactic acid)/poly(glycolic acid)/polethyleneglycol terpolymers, copolymers of polyurethane and poly(lactic acid), copolymers of α-amino acids, copolymers of α-amino acids and caproic acid, copolymers of α-benzyl glutamate and polyethylene glycol, copolymers of poly succinic acid and poly(glycols), polyphosphazene, polyhdroxy-alkanoates, poly(ethylene oxide), poly(ethylene glycol), polypropylene glycol), poly (L-lactic acid), poly(ε-caprolactone), poly(α-amino acids), polyurethanes, poly(vinyl alcohol), poly(vinyl pyrrolidone), poly hydroethyl methacrylate, and poly hydroxyethyl methacrylate.

31. The method of claim 23, wherein the therapeutic agent comprises an anti-cancer agent.

32. The method of claim 23, wherein the energy is selected from the group consisting of ultrasound energy, UV energy, IR energy, visible light, and RF energy.
